(19) United States
(12) Patent Application Publication
(10) Pub. No.: US 2013/0136773 A1
(43) Pub. Date: May 30, 2013

(54) Expandable Placental Membrane and Methods of Making and Storing Same

(71) Applicants: Kenneth L. Horton, Birmingham, AL (US); Gregory J. Yager, Mount Olive, AL (US); Howard P. Wathall, Birmingham, AL (US)

(72) Inventors: Kenneth L. Horton, Birmingham, AL (US); Gregory J. Yager, Mount Olive, AL (US); Howard P. Wathall, Birmingham, AL (US)

(73) Assignee: NuTech Spine, Inc.

(21) Appl. No.: 13/250,096

(22) Filed: Sep. 30, 2011

(51) Int. Cl. A61K 35/50; A61K 9/00; B28B 1/50; C12N 5/071

(52) U.S. Cl. 424/400; 435/325; 424/93.7; 264/154

(57) ABSTRACT

A placental membrane including a plurality of slits for increasing the membrane's capacity to expand. The slits are provided through the membrane and provided in sufficient numbers across the surfaces of the membrane to produce a mesh-like pattern in the membrane. The mesh-like pattern enables the placental membrane to be stretched and therefore increased in length and width. For ease of handling and storage, the expandable placental membrane is removably adhered to a backing, rolled into a cylinder and placed within a capped vial containing a solution of amniotic fluid cells for improving the effectiveness of the membrane.

### FIELD OF THE INVENTION

The present invention is directed to a processed placental membrane. More particularly, the present invention is directed to an expandable placental membrane and methods of making and storing same.

### BACKGROUND OF THE INVENTION

A placenta is an organ surrounding a fetus during gestation. The placenta is composed of, among other tissue, an inner amnion layer facing the fetus and a generally inelastic outer shell or chorion. The placenta anchors the fetus to the uterine wall, allowing nutrient uptake, waste elimination and gas exchange to occur via the mother's blood supply. Additionally, the placenta protects the fetus from an immune response from the mother's body.

From the placenta, a placental membrane composed only of the amnion and chorion can be separated from the other tissues. Clinicians have used intact placental membrane composed of an amnion and a chorion layer in medical procedures since as early as 1910 (Davis, J. S., “Skin Transplantation with a Review of 550 Cases at the Johns Hopkins Hospital,” John Hopkins Med. J., 15:307 (1910)). As an alternative to intact placental membrane, some clinicians separate the amnion from the placental membrane, using only the amnion layer.

Certain characteristics of the placental membrane make it attractive for use by the medical community. Although not an exhaustive list, these characteristics include anti-adhesive, anti-microbial and anti-inflammatory properties, wound protection, epithelialization initiation capacity and pain-reduction. (Mermet, I. et al., “Use of amniotic membrane transplantation in the treatment of venous leg ulcers,” Wound Repair and Regeneration, 15:459 (2007)). Other uses for placental membrane include scaffolding or providing structure for the regrowth of cells and tissue. An important advantage of placental membrane in scaffolding is that the amnion contains an epithelial layer. The epithelial cells derived from this layer are similar to stem cells, allowing the cells to differentiate into cells of the type that surrounds them. Additional cells similar to stem cells are contained in the body of the membrane, and the membrane also contains various growth and trophic factors, such as epidermal, insulin-like and fibroblast growth factors, and high concentrations of hyaluronic acid which may be beneficial in preventing scarring and inflammation and supporting healing. Thus, placental membrane offers a wide-variety of advantages for medical uses.

Although placental membranes possess many benefits and uses, availability of the membranes has limited their use. That is because placental membranes can be collected only from consenting mothers who undergo Cesarean deliveries. Further, on average, a single human, placental membrane weighs approximately 500 grams and is 22 centimeters in length. Thus, the amount of placental membrane generated from a single birth is small. Also, as would be expected, because the supply of placental membranes is relatively small, the cost of placental membranes limits their use only to procedures that surpass a certain price or complexity. For this reason, many conditions that would benefit from the application of placental membranes are not considered for placental membrane treatment. Accordingly, there is a need for means of increasing the effective supply of placental membranes.

### SUMMARY OF THE INVENTION

The present invention is directed to an expandable placental membrane and methods of making and storing same. According to one aspect of the invention, there is provided a material including a placental membrane having an upper surface and a lower surface and a plurality of openings extending through and between the upper surface and the lower surface. The plurality of openings form a mesh-like pattern in the placental membrane that covers the entire upper and lower surfaces of the placental membrane and increases the expandability of the material. The placental membrane includes an amnion layer and a chorion layer, though it is anticipated that either the amnion layer or the chorion layer can be excluded from the membrane.

To improve handling and storage of the placental membrane, an inert, rollable sheet is detachably coupled to the upper surface, the lower surface or both of the placental membrane. The sheet maintains the membrane in a sheet-like form and prevents the membrane from adhering to itself and becoming jumbled, which renders the membrane difficult to apply and may cause it to be torn or damaged. When it is desired to store the placental membrane, the membrane and sheet are rolled into a compact, cylinder-shaped member that can be easily inserted into a vial for storage. A solution of amniotic fluid or cells derived from amniotic fluid may be provided within the vial. Amniotic fluid also contains growth and trophic factors, high concentrations of hyaluronic acid and progenitor cells, such as mesenchymal cells. Research has demonstrated the potential effectiveness of amniotic fluid in fracture healing, nerve regeneration and utilizing progenitor cells to encourage regrowth of bone. By storing the cylindrical-shaped placental membrane within the solution of amniotic fluid or amniotic fluid cells the properties of the amniotic fluid are imparted to or improved in the placental membrane.

According to another aspect of the invention there is provided a method for making a material. The method includes the steps of providing a placental membrane having a first expandability capacity and increasing the first expandability capacity to a second expandability capacity by mechanically processing the membrane. In particular, the increased expandability capacity is provided by forming a plurality of openings in the placental membrane. The plurality of holes can be made by any means known in the art, including, for example, by processing the membrane by hand or with a mesher, which imparts a mesh-like pattern in the placental membrane. A suitable mesher is described in U.S. Pat. No. 5,004,468 to Atkinson, the entire contents of which are incorporated herein in their entirety. Depending on the density, size, shape, pattern and number of openings formed in the placental membrane, the placental membrane can exhibit expansion ratios ranging between 1.2:1 to 6:1, though expansion ratios between 1.5:1 and 3:1 may be preferred. Thus, with the present invention, expansion ratios of 1.25:1; 1.5:1; 1.75:1; 2:1; 2.25:1; 2.5:1; 2.75:1; 3:1; 3.25:1; 3.5:1; 3.75:1; 4:1; 4.25:1; 4.5:1; 4.75:1; 5:1; 5.25:1; 5.5:1; and 5.75:1 are possible.

According to yet another aspect of the invention, there is provided a processed placental membrane including an upper surface, a lower surface, a plurality of openings extending through the upper surface and the lower surface, and a first surface area defined by a continuous, perimeter edge of the placental membrane, the first surface area being expandable to a second surface area defined by the continuous, perimeter edge that is between 20% and 600% greater than the first surface area. Preferably, the plurality of openings form a mesh-like pattern that covers at least 50% of the upper surface and the lower surface of the placental membrane. To increase the ease of handling the membrane, a backing can be detachably coupled to a chorion layer of the placental membrane, the backing covering the entire chorion layer of the placental membrane. To store the membrane, the membrane and backing are rolled into the shape of a cylinder, and the cylinder is placed in a vial, submerged within a solution of amniotic fluid cells within the vial, sealed and frozen.

A further understanding of the nature and advantages of the present invention will be realized by reference to the remaining portions of the specification and the drawings.

### BRIEF DESCRIPTION OF THE DRAWINGS

FIG. 1 is a top plan view of an intact placental membrane in accordance with the prior art.

FIG. 2 is a top plan view of the placental membrane of FIG. 1 meshed in accordance with the present invention.

FIG. 3 is a top plan view of the meshed placental membrane of FIG. 2 expanded.

FIG. 4 is a sectional view of the meshed placental membrane of FIG. 2 adhered to a backing.

FIG. 5 is a perspective view of the meshed placental membrane of FIG. being rolled into the shape of a cylinder.

FIG. 6 is a perspective view of the rolled, meshed placental membrane of FIG. 5 stored within a vial containing a solution of amniotic fluid cells.

FIG. 7 is a side plan view of the meshed placental membrane of FIG. 2 being removed from the backing.

### DETAILED DESCRIPTION OF THE INVENTION

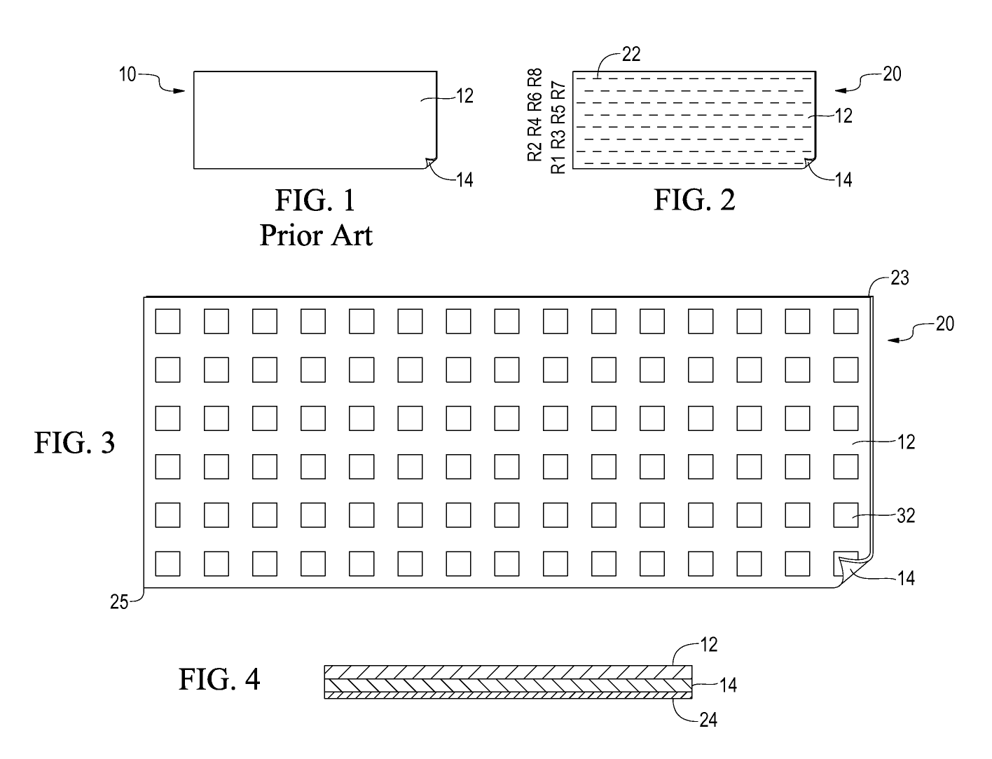

FIG. 1 depicts a prior art placental membrane 10 including an amnion layer 12 and an inelastic chorion layer 14. Placental membrane 10 is of the type of membrane that is commonly used by clinicians in wound healing, cell regeneration and tissue grafting applications.

Placental membrane 10 and similar prior art placental membrane materials are produced from placentas collected from consenting donors in accordance with the Current Good Tissue Practice guidelines promulgated by the U.S. Food and Drug Administration. In particular, soon after the birth of a human infant via a Cesarean section delivery, the intact placenta is retrieved, and the placental membrane is dissected from the placenta. Afterwards, the placental membrane is cleaned of residual blood, placed in a bath of sterile solution, stored on ice and shipped for processing. Once received by the processor, the placental membrane is rinsed to remove any remaining blood clots, and if desired, rinsed further in an antibiotic rinse. The placental membrane is then stored in packs containing a sterile solution or freeze dried.

Prior art placental membrane 10 suffers from several shortcomings. First, since membrane 10 includes generally, inelastic chorion layer 14, the membrane's ability to expand is severely limited. As a result, the ratio of area that can be treated with membrane 10 to the surface area of either the upper or lower surface of the membrane is about 1:1. Second, the intact placental membrane 10 is essentially impermeable which, when in use, limits the migration of cells and molecules across membrane 10 which, in turn, can limit, for example, the desired growth of blood vessels across the membrane. Consequently, membrane 10 may actually impair healing in certain instances. Nonetheless, current medical procedures involving the application of placental membrane call for maintaining the integrity of the membrane. Third, the means of storing placental membrane 10 makes handling of the membrane tedious since the membrane is thin, limp and tends to adhere to itself. When retrieved from a liquid storage solution, membrane 10 can appear as an amorphous clump of tissue. This makes locating the edges of membrane 10 difficult and increases the likelihood that the process of spreading out the membrane into a flat sheet will cause tearing of the membrane. This process also consumes valuable surgical time.

FIGS. 2 through 4 depict an expandable, porous placental membrane material 20 that is prepared from placental membrane 10 in accordance with a preferred embodiment of the present invention. Placental membrane material 20 is processed in a manner that allows it to expand to cover more than six times the treatment area than prior art membrane 10. This is accomplished by forming a plurality of openings through placental membrane 10 and imparting to the membrane a mesh-like pattern. The resulting expandable, porous placental membrane material 20 enables material 20 to stretch along its length and width thereby increasing the perimeter of material 20 and the amount of surface area it can cover when placed on or in a wound or the like.

More particularly, referring to FIG. 2, placental membrane material 20 includes a plurality of elongated slits 22 which in combination provide material 20 with a mesh-like appearance. Slits 22 extend between and through amnion layer 12 and chorion layer 14 and are formed by processing placental membrane 10 with a mesher, for example, as described in U.S. Pat. Nos. 6,063,094; 5,004,468; 3,640,279; 3,472,228 and 3,358,688. Depending on the mesher settings, and more particularly, the arrangement and number of the cutting portions of the mesher, slits 22 can vary in size, density and orientation. By varying slit 22 sizes, densities and orientation the capacity for placental membrane material 20 to expand can be controlled. Preferably, slits 22 are arranged in a series of substantially parallel rows R1 through R8 that extend longitudinally along the length of material 10 with adjacent slits 22 being staggered or offset and slits 22 of alternate rows R1, R3, R5 and R7 and alternate rows R2, R4, R6 and R8 being aligned. Using a mesher, as opposed to forming slits 22 by hand, provides a high through-put method of manufacturing placental membrane material 20.

Arranged as described above, slits 22 provide within placental membrane material 20 a mesh-like arrangement, the mesh-like arrangement imparting to placental membrane material 20 an increased capacity to expand, mostly along the width of material 20. In particular, referring to FIG. 3, upon applying outward force to placental membrane material 20, for example, by grasping two opposing corners 23, 25 of material 20 with forceps and pulling placental membrane material 20 outwardly, the distance between the edges of the slits 22 moves apart to expand the width of material 20. As the material expands, slits 22 of placental membrane material 20 widen due to lateral and/or vertical movement of slit edges relative to one another, thus expanding slits 22 created by the mesher. In this manner, the perimeter of material 20 can is increased.

The mesh-like pattern created by expanding slits 22 and, in turn, the perimeter of material 20, depends on the manner in which meshed placental membrane material 20 is manipulated by the clinician. The mesh pattern may be square shaped 32 as seen in FIG. 3 or hexagonal shape (not pictured). Alternatively, it is anticipated that the openings formed through placental membrane material can be shaped other than as slit 22, and thus the mesh-like pattern may include shapes other than squares. For example, the openings may be formed as two intersecting slits such as cross-shaped or X-shaped if it is desired to widen, as well as substantially lengthen material 20. Additionally, it is anticipated that the openings can be L-shaped, H-shaped, or Z-shaped since, like cross-shaped or X-shaped openings, these openings will allow for substantial lengthening and widening of material 20.

In addition to increasing the capacity for material 20 to expand, formation of slits 22 in the material imparts a porosity to material 10 that is not found in placental membrane 10 by virtue of the impermeable nature of the intact placental membrane 10. By providing pathways through material 20, wound draining is facilitated and movement of molecules and cells across placental membrane material 20 enabled. These properties are expected to increase the effectiveness of material 20 in certain wound healing and grafting applications.

Preferably, slits 22 are dispersed over the entire surface of material 20 in order to maximize expandability of the material; however, is anticipated that there may be applications where expandability or porosity of material 20 may be desired for only certain portions of the material. In those instances, slits 22 may be provided in only a fraction of the material or limited only to certain areas of the material such as around the perimeter of the material, in a central portion of the material, or within a top, bottom, left or right half of the material.

The addition of slits 22 to placental membrane material 20 can negatively affect the ability to handle the material since the existence of slits 22 increases the flaccidness of the membrane. Also, with the addition of slits 22, material 20 is more delicate than placental membrane 10 and therefore more prone to tearing. These difficulties manifest when attempting to remove placental membrane material 20 from a storage container and spreading out the material into the form of a sheet for application to a patient. Accordingly, there is needed a means of storing placental membrane material 20 in a manner that allows a clinician to easily retrieve the material from a storage container and spread out the material for application to a patient.

Referring to FIGS. 4 through 7, there is depicted a means for compactly storing and maintaining placental membrane material 20 in a flat, un-jumbled sheet-like arrangement. As illustrated, to maintain placental membrane material 20 in a sheet-like arrangement, material 20 is spread out flat and chorion layer 14 is adhered directly to a flat, flexible, inert backing 24 so that the entire chorion layer 14 is covered. Alternatively, backing 24 can be adhered to amnion layer 12, or an additional backing can be used to sandwich material 20 between two backing layers. Use of two backings 24 is best utilized if placental membrane material 20 is to be stored in a flat arrangement, for example, in a flexible, plastic bag or the like. Backing may be applied with the membrane in an expanded or unexpanded configuration. Exemplary backing materials include polyvinyl chloride and nitrocellulose paper.

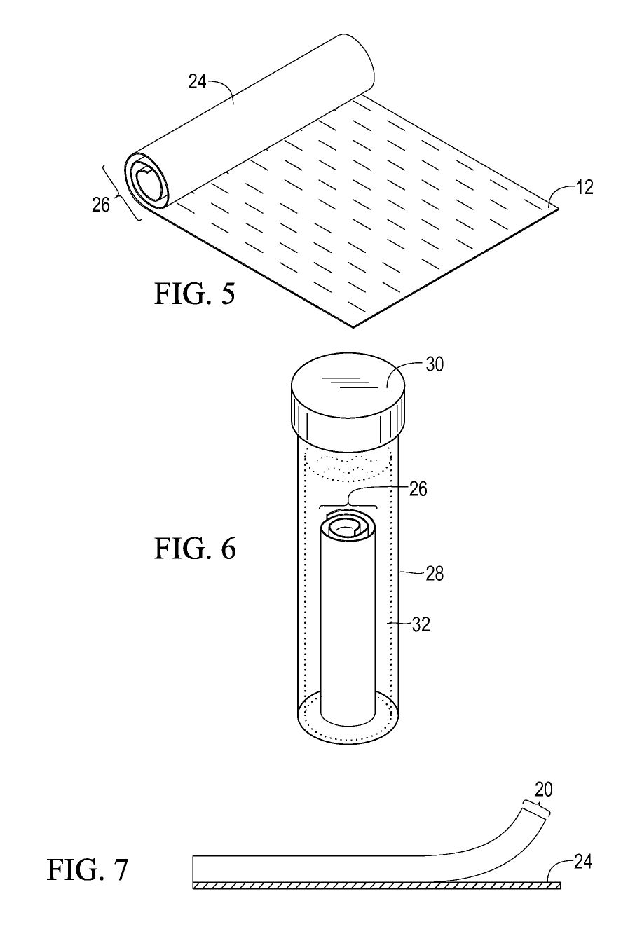

Referring to FIG. 5, once removably adhered to backing 24, backing 24 and placental membrane material 20 are rolled into a compact, cylindrically-shaped member 26. Preferably, backing 24 and material 20 are arranged in cylindrically-shaped member 26 in such a way that backing 24 is located on the exterior of cylindrically-shaped member 26. This way, placental membrane material 20 is protected from contacting and thus sticking to anything other than backing 24. By rolling material 20 and backing 24 into cylindrically-shaped member 26, the membrane is compressed between two layers of backing 24. Compression of placental membrane material 20 in this manner, prevents or inhibits the shrinking of material 20 which is often seen during the freeze drying of prior art placental membrane 10.

Referring to FIG. 6, after cylindrically-shaped member 26 is formed, it can be stored in a cylindrical, freezable, sealable vial 28 having a removable cap 30. Preferably, vial 28 includes a cell solution 32 derived from amniotic fluid collected from the donor of placental membrane 10. Cell solution 32 is a prepared by separating cells and other materials in the amniotic fluid from the amniotic fluid by centrifugation and suspending the retained pellet containing the cells and other materials in a sterile, inert solution. By submerging and storing placental membrane material 20 in cell solution 32, many of the beneficial properties of the cell solution are imparted to or are improved in material 20. As a result, material 20 is expected to exhibit improved effectiveness such as improved fracture healing, nerve regeneration and cell regrowth.

Referring to FIG. 7, following production and storage of placental membrane 20 in vial 28, when material 20 is to be used, cylindrically-shaped member 26 is retrieved from vial 28 and un-rolled. Once flattened into a sheet, placental membrane material 20 can be removed from backing 24 in a manner that maintains the integrity and shape of material 20. Typically, this is accomplished by grasping two corners of material 20 with forceps and lifting material 20 slowly from backing 24. With placental membrane material 20 removed from backing 24 and maintaining a generally flat configuration, the clinician is able to easily expand material 20 to the desired size, and place material 20 over the treatment area. In some instances, prior to removing material 20 from backing 24, placental membrane material 20 can be cut into a desired shape or size since doing so is made easier by its support on backing 24. Depending on the density, size, shape and number of openings formed in placental membrane material 20, the clinician can expand the material to cover up to three times or more treatment surface area than placental membrane 10.

As will be understood by those familiar with the art, the present invention may be embodied in other specific forms without departing from the spirit or essential characteristics thereof. Accordingly, the disclosures and descriptions herein are intended to be illustrative, but not limiting, of the scope of the invention which is set forth in the following claims.

1-28. (canceled)

29. A method for making a material comprising,
providing a placental membrane having a first expandability capacity, and
increasing the first expandability capacity to a second expandability capacity by mechanically processing the placental membrane,
wherein the second expandability capacity of the placental membrane is between 20% to 50% greater than the first expandability capacity.

30. A method for making a material comprising,
providing a placental membrane having a first expandability capacity, and
increasing the first expandability capacity to a second expandability capacity by mechanically processing the placental membrane,
wherein the second expandability capacity of the placental membrane is between 50% to 100% greater than the first expandability capacity.

31. A method for making a material comprising,
providing a placental membrane having a first expandability capacity, and
increasing the first expandability capacity to a second expandability capacity by mechanically processing the placental membrane,
wherein the second expandability capacity of the placental membrane is between 100% to 300% greater than the first expandability capacity.

32-35. (canceled)

36. The method according to claim 29 wherein the mechanical processing includes forming a plurality of openings in the placental membrane.

37. The method according to claim 29 wherein the mechanical processing includes passing the placental membrane through a mesher.

38. The method according to claim 29 wherein the mechanical processing imparts a mesh-like pattern in the placental membrane.

39. The method according to claim 36 further comprising limiting the plurality of openings to a specific portion of the placental membrane, wherein the specific portion is determined by identifying a medical treatment for which the placental membrane will be used and tailoring the specific portion to provide the placental membrane with a desired expandability and a desired porosity in desired locations across the placental membrane for increasing an effectiveness of the placental membrane when used in the medical treatment.

40. The method according to claim 29 further comprising covering a surface of the placental membrane with an inert sheet and rolling the inert sheet and the placental membrane into a cylinder.

41. The method according to claim 40 further comprising at least partially submerging the cylinder within a solution of amniotic fluid cells.

42. The method according to claim 41 further comprising placing the cylinder in a vial.

43. The method according to claim 38 wherein the mesh-like pattern covers at least 50% of an upper surface and a lower surface of the placental membrane.

44. The method according to claim 36 wherein the plurality of openings are in the form of slits.

45. The method according to claim 36 wherein at least one opening of the plurality of openings is selected from a group consisting of a cross-shaped opening, an X-shaped opening, an L-shaped opening, an H-shaped opening and a Z-shaped opening.

46. The method according to claim 30 wherein the mechanical processing includes forming a plurality of openings in the placental membrane.

47. The method according to claim 30 wherein the mechanical processing includes passing the placental membrane through a mesher.

48. The method according to claim 30 wherein the mechanical processing imparts a mesh-like pattern in the placental membrane.

49. The method according to claim 46 further comprising limiting the plurality of openings to a specific portion of the placental membrane, wherein the specific portion is determined by identifying a medical treatment for which the placental membrane will be used and tailoring the specific portion to provide the placental membrane with a desired expandability and a desired porosity in desired locations across the placental membrane for increasing an effectiveness of the placental membrane when used in the medical treatment.

50. The method according to claim 30 further comprising covering a surface of the placental membrane with an inert sheet and rolling the inert sheet and the placental membrane into a cylinder.

51. The method according to claim 50 further comprising at least partially submerging the cylinder within a solution of amniotic fluid cells.

52. The method according to claim 48 wherein the mesh-like pattern covers at least 50% of an upper surface and a lower surface of the placental membrane.

53. The method according to claim 46 wherein the plurality of openings are in the form of slits.

54. The method according to claim 46 wherein at least one opening of the plurality of openings is selected from a group consisting of a cross-shaped opening, an X-shaped opening, an L-shaped opening, an H-shaped opening and a Z-shaped opening.

55. The method according to claim 31 wherein the mechanical processing includes forming a plurality of openings in the placental membrane.

56. The method according to claim 31 wherein the mechanical processing includes passing the placental membrane through a mesher.

57. The method according to claim 31 wherein the mechanical processing imparts a mesh-like pattern in the placental membrane.

58. The method according to claim 31 further comprising covering a surface of the placental membrane with an inert sheet and rolling the inert sheet and the placental membrane into a cylinder.

59. The method according to claim 58 further comprising at least partially submerging the cylinder within a solution of amniotic fluid cells.
